(19) United States
(12) Patent Application Publication
(10) Pub. No.: US 2020/0246085 A1
(43) Pub. Date: Aug. 6, 2020

(54) OPTICAL REGISTATION OF A REMOTE CENTER OF MOTION ROBOT

(71) Applicant: KONINKLIJKE PHILIPS N.V., EINDHOVEN (NL)

(72) Inventors: David Paul NOONAN, New York, NY (US); Aleksandra POPOVIC, BOSTON, MA (US)

(21) Appl. No.: 15/762,757

(22) PCT Filed: Sep. 26, 2016
(86) PCT No.: PCT/IB2016/055743

§ 371 Date: Mar. 23, 2018

Related U.S. Application Data
(60) Provisional application No. 62/233,664, filed on Sep. 28, 2015.

(51) Int. Cl. A61B 34/30; A61B 34/10; A61B 90/13

(52) U.S. Cl. CPC A61B 34/30; A61B 34/10; A61B 2090/3966; A61B 2034/107; A61B 90/13

(57) ABSTRACT

A robotic surgical system for a minimally invasive procedure involving a planned tool trajectory through a planned incision point into a patient. The robotic surgical system employs an optical end-effector (50) (e.g., a laser point or an endoscope), a RCM robot (40) (e.g., a concentric arc robot), and a robot controller (60). In operation, the robot controller (60) controls an optical pointing by the RCM robot (40) of the optical end-effector (50) to one or more markers attached to the patient, and further controls an axial alignment by the RCM robot (40) of the optical end-effector (50) to the planned tool trajectory as illustrated within a volume image of the patient based on a registration of the remote center-of-motion to the planned incision point as illustrated within the volume image of the patient derived from the optical pointing.

### FIELD OF THE INVENTION

The present disclosure generally relates to minimally invasive procedures requiring an interventional tool to be inserted into a patient along a defined tool trajectory through at a specific incision point, particularly minimally invasive neurosurgical procedures (e.g. a biopsy). The present disclosure more particularly relates a registration of remote center-of-motion (“RCM”) by a remote center-of-motion robot to planned incision point into the patient for accurately positioning and orienting the interventional tool along a planned tool trajectory.

### BACKGROUND OF THE INVENTION

Image guided brain biopsy allows surgeons to accurately target deep seated brain lesions in a minimally invasive manner. Specifically, the patient's head is immobilized and registered to a pre-operative imaging scan (e.g., CT, MRI, US, etc.) using a tracking and localization system (e.g., optical, electromagnetic, mechanical or combination thereof). Typically, such registration is performed using markers placed on the skull of the patient and/or tracked manual pointers. Based on the known position as illustrated within the pre-operative imaging scan of the brain lesion with respect to the tracking system, an appropriate location of an incision point into the patient is identified by the surgeon for the biopsy. The surgeon then manually aligns the insertion angles of the tracked biopsy needle based on feedback from the image guided tracking system. As the needle is inserted by the surgeon, the image guidance tracking system confirms the needle trajectory and identifies when the correct insertion depth has been reached. To this ends, image guidance systems as known in the art may provide a mechanical needle guide which the surgeon aligns to the planned tool trajectory prior to needle insertion.

Even when using such image guidance, and assuming an accurate registration of the pre-operative imaging scan to the tracking system has been achieved, surgeons must perform a five-degree (5°) of freedom alignment in free space. Thus, user error in registration and/or needle alignment can lead to incorrect incision locations, and/or the interventional tool missing the target brain lesion inside the patient's head.

### SUMMARY OF THE INVENTION

The present disclosure provides inventions using a robot mounted optical end-effector (e.g., laser pointer or an endoscope) for registering a remote center-of-motion (“RCM”) robot to an image of the patient during a minimally invasive procedure (e.g., a minimally invasive neurosurgical procedure). By registering the image of the patient to the RCM robot, an exact position and orientation of a planned tool trajectory for performing the procedure is automatically defined in an accurate manner. This in turn allows for the surgeon to deploy the interventional tool in a precise, controlled manner with minimal risk of human error.

One form of the inventions of the present disclosure is a robotic surgical system for a minimally invasive procedure involving a planned tool trajectory through a planned incision point into a patient.

The robotic surgical system employs an optical end-effector (e.g., a laser point or an endoscope) and a RCM robot for rotating the optical end-effector about a remote center-of-motion defined by a structural configuration of the RCM robot.

The robotic surgical system further employs a robot controller for controlling an optical pointing by the RCM robot of the optical end-effector to one or more markers attached to the patient, and for controlling an axial alignment by the RCE robot of the optical end-effector to the planned tool trajectory as illustrated within a volume image of the patient based on a registration of the remote center-of-motion to the planned incision point as illustrated within the volume image of the patient derived from an optical pointing by the RCM robot of the optical end-effector to the registration marker(s) attached to the patient.

A second form of the inventions of the present disclosure is a robotic surgical method for a minimally invasive procedure involving a planned tool trajectory through a planned incision point into a patient.

The robotic surgical method involves a RCM robot optically pointing an optical end-effector to one or more markers attached to the patient, and a registration module deriving a registration of a remote center-of-motion to the planned incision point as illustrated within a volume image of the patient from the optical pointing by the RCM robot to the optical end-effector to the marker(s) attached to the patient,

The remote center-of-motion is defined by a structural configuration of the RCM robot.

The robotic surgical method further involves the RCM robot axially aligning the optical end-effector to the planned tool trajectory as illustrated within the volume image of the patient based on the registration by the registration module of the remote center-of-motion to the planned incision point as illustrated within the volume image of the patient.

For purposes of the inventions of the present disclosure, terms of the art including, but not limited to, “planned tool trajectory”, “planned incision point”, “end-effector”, “remote center-of-motion”, “robot”, “marker” and “volume image”, are to be interpreted as understood in the art of the present disclosure and as exemplarily described herein.

More particularly, for purposes of the inventions of the present disclosure, the term “optical end-effector” broadly encompasses any device serving as an end-effector of a robot and having optical capabilities for emitting and/or receiving any form of radiation, and the term “RCM robot” broadly encompasses any robot having a structural configuration defining a remote center-of-motion whereby the robot or a portion thereof is rotatable about a point spatially fixed from the robot. Examples of an optical end-effector include, but are not limited to, any type of laser pointer and endoscope as known in the art and exemplarily described herein, and an example of a RCM robot includes, but is not limited to, any type of concentric arc robot as known in the art and exemplarily described herein.

For purposes of the inventions of the present disclosure, the term “controller” broadly encompasses all structural configurations of an application specific main board or an application specific integrated circuit housed within or linked to a workstation for controlling an application of various inventive principles of the present disclosure as subsequently described herein. The structural configuration of the controller may include, but is not limited to, processor(s), computer-usable/computer readable storage medium(s), an operating system, application module(s), peripheral device controller(s), slot(s) and port(s).

Examples of the workstation include, but are not limited to, an assembly of one or more computing devices (e.g., a client computer, a desktop and a tablet), a display/monitor, and one or more input devices (e.g., a keyboard, joysticks and mouse).

For purposes of the inventions of the present disclosure, the term “application module” broadly encompasses a component of the controller consisting of an electronic circuit and/or an executable program (e.g., executable software and/firmware) for executing a specific application.

For purposes of the inventions of the present disclosure, descriptive labeling of the a controller herein as a “robot” controller and an “imaging” controller serves to identify a particular controller as described and claimed herein without specifying or implying any additional limitation to the term “controller”.

Similarly, for purposes of the inventions of the present disclosure, descriptive labeling of an application module herein as a “servo control” module and a “registration control” module serves to identify a particular application module as described and claimed herein without specifying or implying any additional limitation to the term “application module”.

The foregoing forms and other forms of the present disclosure as well as various features and advantages of the present disclosure will become further apparent from the following detailed description of various embodiments of the present disclosure read in conjunction with the accompanying drawings. The detailed description and drawings are merely illustrative of the present disclosure rather than limiting, the scope of the present disclosure being defined by the appended claims and equivalents thereof.

### BRIEF DESCRIPTION OF THE DRAWINGS

FIG. 1 illustrates a first exemplary embodiment of a minimally invasive neurosurgical procedure in accordance with the inventive principles of the present disclosure.

FIG. 2 illustrates flowcharts representative of an exemplary embodiment of an robotic image guidance method in accordance with the inventive principles of the present disclosure.

FIGS. 3A-3D illustrate exemplary embodiments of registration markers in accordance with the inventive principles of the present disclosure.

FIGS. 4A-4G illustrate an exemplary registration of a RCM robot to a patient in accordance with the inventive principles of the present disclosure.

FIG. 5 illustrates a second exemplary embodiment of a minimally invasive neurosurgical procedure in accordance with the inventive principles of the present disclosure.

FIG. 6 illustrates a third exemplary embodiment of a minimally invasive neurosurgical procedure in accordance with the inventive principles of the present disclosure.

FIGS. 7A and 7B illustrate exemplary embodiments of workstations in accordance with the inventive principles of the present disclosure.

### DETAILED DESCRIPTION OF THE PREFERRED EMBODIMENTS

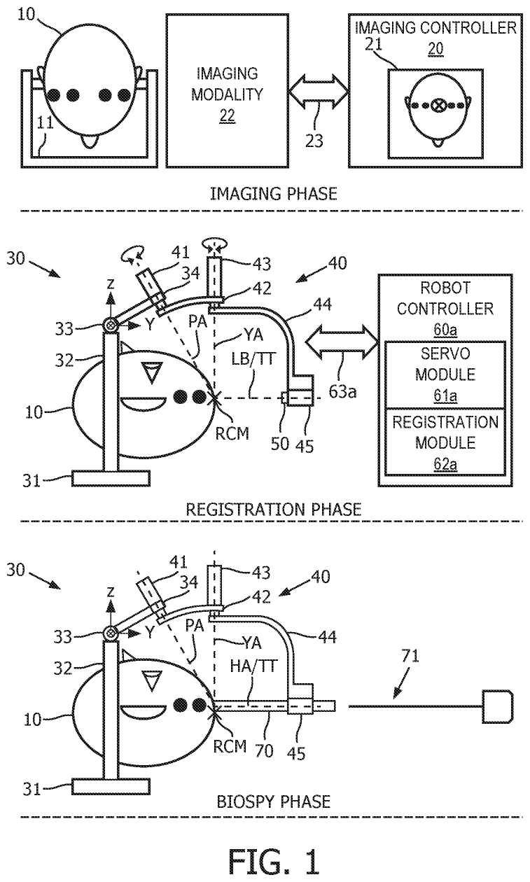

To facilitate an understanding of the present disclosure, the following description of FIG. 1 teaches basic inventive principles of using a robot mounted laser pointer for registering a remote center-of-motion (“RCM”) robot to an image of a patient during a minimally invasive neurosurgical procedure. From this description, those having ordinary skill in the art will appreciate how to apply the inventive principles of the present disclosure to various optical end-effectors for registering a RCM robot to an image of a patient during any type of minimally invasive procedure.

Referring to FIG. 1, an imaging phase of a minimally invasive biopsy utilizes an imaging controller 20, an imaging modality 22 (e.g., a CT, MRI or US imaging modality), and a communication path 23 (e.g., wired/wireless connection(s)) between imaging controller 20 and imaging modality 22. Generally in operation, the image phase of the minimally invasive biopsy involves imaging controller 20 controlling a display of a generation by imaging modality 22 as known in the art of a volume image 21 illustrative of markers (symbolized as black dots) attached to a head of a patient 10 immobilized by a clamp 11. Upon the imaging, imaging controller 22a further controls a user planning within volume image 21 as known in the art a location of an incision point on the head of patient 10 (symbolized by a circle between the markers of volume image 21) and a tool trajectory through the incision point for purposes of reaching a target lesion within the head of patient 10 (symbolized by an X within the marker circle of volume 21 representing a tool trajectory perpendicular to the incision point).

Still referring to FIG. 1, a registration phase of the minimally invasive biopsy utilizes a robot platform 30, a RCM robot in the form of a concentric arc robot 40, an optical end-effector in the form a laser pointer 50, a robot controller 60a, and a communication path 63a (e.g., wire/wireless connection(s)) between robot controller 60a and a concentric arc robot 40, and between robot controller 60a and active embodiments of robot platform 30.

For purposes of the inventions of the present disclosure, the term “robot platform” broadly encompasses any platform structurally configured for moving a RCM robot of the present disclosure within a Cartersian coordinate system whereby a remote center-of-motion of may be moved to a desired point within the Cartesian coordinate system.

For the embodiment of FIG. 1, robot platform 30 employs a base 31 (connectable to a bed rail or other stable location within an operating space) and a post 32 stationary relative to base 31, or translatable, pivotable and/or extendable relative to base 31. Robot platform 30 further employs a robot holding arm 34, and a joint 33 interconnecting robot holding arm 34 to post 32 whereby robot holding arm 34 is translatable, pivotable and/or extendable relative to post 32 within the Cartesian coordinate system.

In practice, robot platform 30 may be passive in terms of a manual manipulation of post 32 and/or robot holding arm 34, or active in terms of a motorized post 32 and/or a motorized joint 33 controlled by robot controller 60a for commanding command a translation, a pivot and/or an extension of base 31 and/or robot holding arm 34 via communication path 63a. For both passive and active embodiments of robot platform 30, post 32 and/or joint 33 may include encoders (not shown) for generating encoded signals informative of a pose of post 32 relative to base 31 and/or of a pose of robot holding arm 34 within the Cartesian coordinate system whereby robot controller 60a may track post 32 and/or robot holding arm 34.

Concentric arc robot 40 employs a pitch arc 42 mechanically coupled to a pitch actuator 41, and mechanically coupled to or physically integrated with a yaw actuator 43. concentric arc robot 40 further includes a yaw arc 44 mechanically coupled to yaw actuator 43, and mechanically coupled to or physically integrated with an end-effector holder 45.

Pitch actuator 41 includes an encoded motor (not shown) controllable by robot controller 60a via communication path 63a for selectively actuating pitch actuator 41 to simultaneously revolve pitch arc 42, yaw actuator 43, yaw arc 44, end-effector holder 45 and laser pointer 50 about a pitch axis PA of pitch actuator 41 as symbolized by the bidirectional arrow encircling pitch axis PA.

Yaw actuator 43 includes an encoded motor (not shown) controllable by robot controller 60a via communication path 63a for selectively actuating yaw actuator 43 to thereby revolve yaw actuator 43, yaw arc 44, end-effector holder 45 and laser pointer 50 about a yaw axis YA of yaw actuator 41 as symbolized by the directional arrow encircling yaw axis YA.

End-effector holder 45 is structurally configured as known in the art to hold laser pointer 50 whereby a laser beam LB emitted by laser pointer 50a is aligned with a longitudinal axis of end-effector holder 45.

As known in the art, a relative orientation of pitch actuator 41, yaw actuator 43 and end-effector holder 45 defines a remote center-of-motion RCM as an intersection of pitch axis PA, yaw axis YA and the end-effector axis (represented by laser beam LB). Based on encoded signals generated by active embodiments of robot platform 30, robot controller 60a executes a servo module 61a as known in the art for strategically positioning remote center-of-motion RCM relative to the head of patient 10. Based on encoded signals generated by pitch actuator 41 and yaw actuator 43, robot controller 60a executes servo module 61a as known in the art for tactically orienting laser pointer 50 relative to the markers attached to the head of the patient 10.

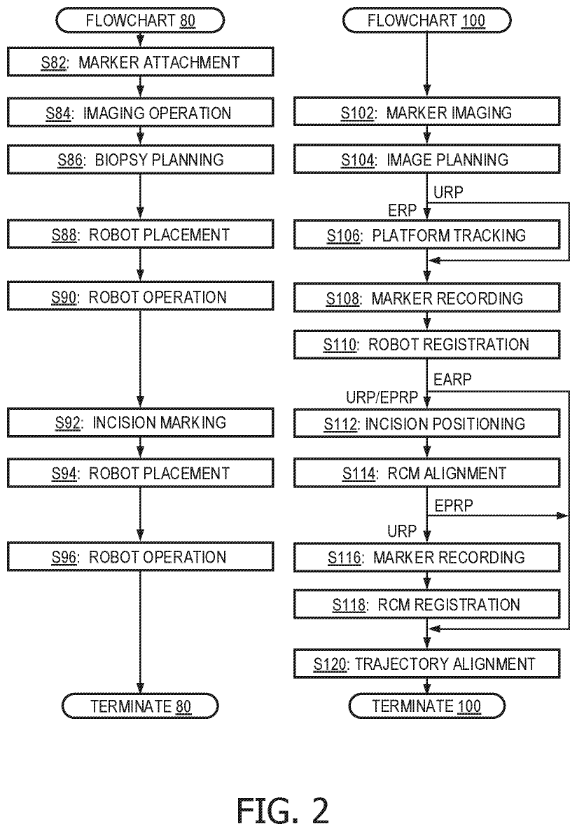

Generally in operation, the registration phase of the minimally invasive biopsy involves robot controller 60a executes a registration module 62a for registering remote center-of-motion RCM to the location of the incision point within volume image 21 of the head of patient 10 whereby laser beam LA is aligned with the tool trajectory TT planned during the imaging phase. A more detailed description of registration module 62a will be provided with the description of FIG. 2.

Still referring to FIG. 1, a biopsy phase of the minimally invasive biopsy involves a removal of laser pointer 50 from end effector holder 45 and an insertion of a tool guide 70 within end-effector holder 45. Based on the registered alignment of laser beam LB to the planned tool trajectory TT during the registration phase, a biopsy needle 71 may be deployed in a precise, controlled manner as measured by tool guide 70 to reach the target lesion within the head of patient 10.

To facilitate a further understanding of registration module 62a, the following is a description of an imaging phase and registration phase of an exemplary robotic image guidance method of the present disclosure as shown in FIG. 2 in the context of the minimally invasive biopsy shown in FIG. 1. From this description, those having ordinary skill in the art will appreciate how to apply the inventive principles of the present disclosure for embodying a registration module applicable to any particular minimally invasive procedure and any particular structural configuration of a RCM robot and a robot platform of the present disclosure.

FIG. 2 illustrates a flowchart 80 represents actions performed by a surgeon of the exemplary robotic image guidance method of the present disclosure, and a flowchart 100 represents actions performed by controllers of the exemplary robotic image guidance method of the present disclosure.

Referring to FIGS. 1 and 2, during the imaging phase of FIG. 1, a stage S82 of flowchart 80 encompasses a surgeon attaching radio opaque markers to patient 10 as known in the art, and a stage S84 of flowchart 80 encompasses the surgeon interfacing with imaging controller 20 whereby imaging modality 22 as controlled by imaging controller 20 during a stage S102 of flowchart 100 generates volume image 21 illustrative of the opaque markers attached to the head of patient 10. In practice, the markers may have an identical configuration, or each marker have a unique shapes to facilitate individual identification of the markets within volume image 21.

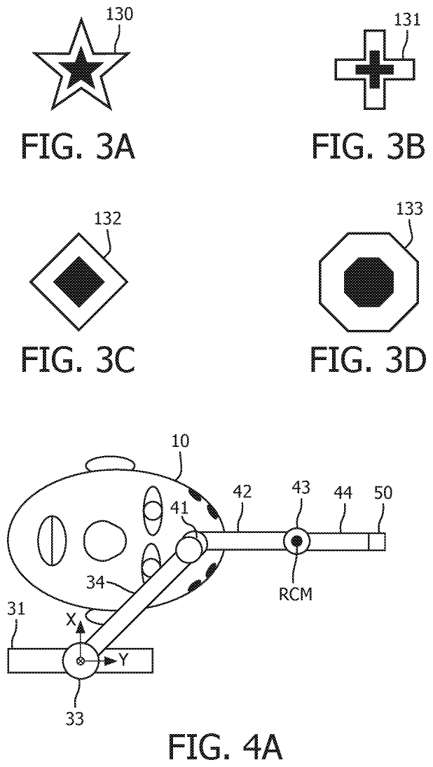

For example, FIG. 3A illustrates a radio opaque marker 130 having a star shape, FIG. 3B illustrates a radio opaque marker 131 having a cross shape, FIG. 3C illustrates a radio opaque marker 132 having a diamond shape, and FIG. 3D illustrates a radio opaque marker 133 having an hexagonal shape.

Referring back to FIGS. 1 and 2, subsequent to imaging stages S84 and S102, a stage S86 of flowchart 80 encompasses the surgeon interfacing with image controller 20 during a stage S104 of flowchart 100 for planning the location of the incision point into the head of patient 20 within volume image 21 as known in the art and for planning the tool trajectory through the incision point to reach the lesion in the head of patient 20 within the volume image 21 as known in the art.

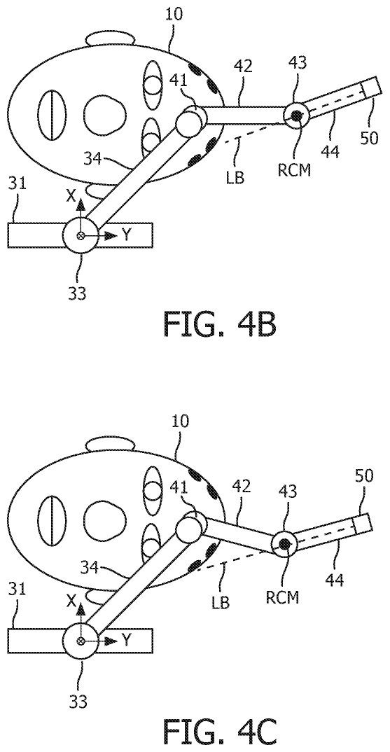

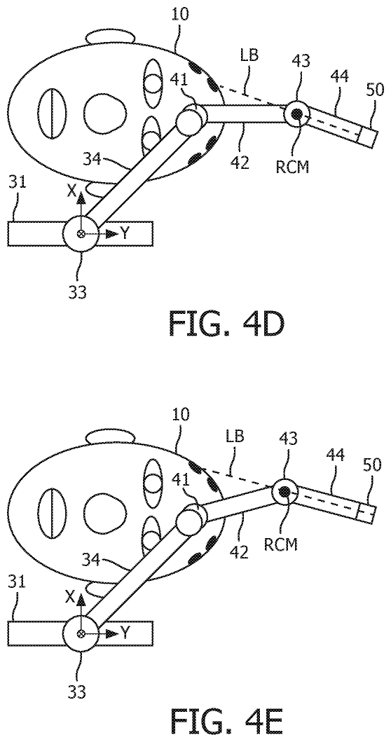

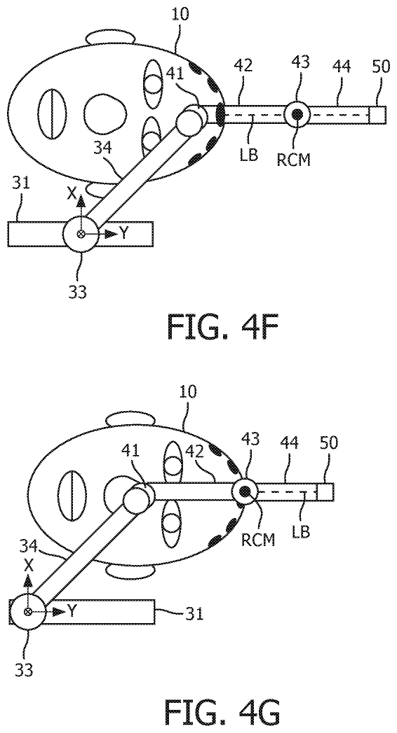

Still referring to FIGS. 1 and 2, at the onset of the registration phase of FIG. 1, a stage S88 of flowchart 80 encompasses the surgeon manually manipulating a passive robot platform 30 or interfacing with servo module 61a for an active robot platform 30 to arbitrarily position remote center-of-motion RCM spatially from the head of patient 10 as exemplarily shown in FIG. 4A. For un-encoded passive or active embodiments of robot platform 30 (“URP”), registration module 62a is not informed via encoded signals of the aforementioned manual or servo control of robot platform 30 and proceeds to a stage S108 of flowchart 100. For encoded passive or active embodiments of robot platform 30 (“ERP”), a stage S104 of flowchart 100 encompasses registration module 62a being informed via encoded signals of the arbitrary positioning of the remote center-of-motion RCM spatially from the head of patient 10 during stage S88 whereby registration module 62a initiates a tracking of robot platform 30.

Subsequent a completion of stage S88, a stage S90 of flowchart 80 encompasses the surgeon interfacing with servo module 61a for sequentially centering laser beam LB of laser pointer 50 on each marker whereby a stage S108 of flowchart 100 encompasses servo module 61a or registration module 62a recording an encoded position of pitch actuator 41 and yaw actuator 42 for a centering of laser beam LB on each marker.

For example, FIG. 4B illustrates a servo control by servo module 61a of yaw actuator 42 for centering laser beam LB on a first marker whereby servo module 61a or registration module 62a records an encoded position of pitch actuator 41 and yaw actuator 42 corresponding to the centering of laser beam LB on the first marker.

FIG. 4C illustrates a servo control by servo module 61a of pitch actuator 41 for centering laser beam LB on a second marker whereby servo module 61a or registration module 62a records an encoded position of pitch actuator 41 and yaw actuator 42 corresponding to the centering of laser beam LB on the second marker.

FIG. 4D illustrates a servo control by servo module 61a of pitch actuator 41 and yaw actuator 42 for centering laser beam LB on a third marker whereby servo module 61a or registration module 62a records an encoded position of pitch actuator 41 and yaw actuator 42 corresponding to the centering of laser beam LB on the third marker.

And, FIG. 4E illustrates a servo control by servo module 61a of pitch actuator 41 for centering laser beam LB on a fourth marker whereby servo module 61a or registration module 62a records an encoded position of pitch actuator 41 and yaw actuator 42 corresponding to the centering of laser beam LB on the fourth marker.

Subsequent to a completion of stage S108, a stage S110 of flowchart 100 encompasses registration module 62a processing the recorded encoded positions of pitch actuator 41 and yaw actuator 42 as known in the art to register concentric arc robot 40 to the markers and planned incision location as illustrated in volume image 21.

Based on the registration of stage S110, for un-encoded passive or active embodiments of robot platform 30 (“URP”), a stage S112 of flowchart 100 encompasses an automatic servo control via servo module 61a of pitch actuator 41 and/or yaw actuator 43 as needed to center laser beam LB on the planned incision location as exemplarily shown in FIG. 4F with a symbolically black dot.

Subsequent to a completion of stage S110, a stage S92 of flowchart 80 encompasses the surgeon marking the incision location on the head of patient as indicated by laser beam LB during stage S112, and a stage S94 of flowchart 80 encompasses the surgeon manually manipulating a passive robot platform 30 or interfacing with servo module 61a for an active robot platform 30 to align the remote center-of-motion RCM with the incision marker during a stage S114 of flowchart 100 as exemplarily shown in FIG. 4G. Registration module 62a provides a graphical and/or textual confirmation of the alignment.

Subsequent to a completion of stages S94 and S114, a stage S96 of flowchart 90 encompasses the surgeon interfacing with servo module 61a for sequentially centering laser beam LB of laser pointer 50 on each marker including the incision marker whereby a stage S114 of flowchart 100 encompasses servo module 61a or registration module 62a recording an encoded position of pitch actuator 41 and yaw actuator 42 for a centering of laser beam LB on each marker. FIGS. 4B-4E are exemplary of stage S114 with the remote center-of-motion being aligned with the incision marker as opposed to being spaced from the head of patient 10.

Subsequent to the centering of laser beam LB on each marker, a stage S118 of flowchart 100 encompasses registration module 62a processing the recorded encoded positions of pitch actuator 41 and yaw actuator 42 as known in the art to register the remote center-of-motion RCM to incision marker as illustrated in volume image 21. Based on the registration of stage S118, a stage S120 of flowchart 100 encompasses an automatic servo control via servo module 61a of pitch actuator 41 and/or yaw actuator 43 as needed to axially align laser beam LB with the planned tool trajectory TT as shown in FIG. 1.

Based on the registration of stage S110, for encoded passive embodiments of robot platform 30 (“EPRP”), stage S92 of flowchart 80 again encompasses the surgeon marking the incision location on the head of patient as indicated by laser beam LB during stage S112, and stage S94 of flowchart 80 again encompasses the surgeon manually manipulating a passive robot platform 30 to align the remote center-of-motion RCM with the incision marker during stage S114 of flowchart 100 as exemplarily shown in FIG. 4G. Registration module 62a provides a graphical and/or textual confirmation of the alignment.

With the encoding tracking of stage S106, a registration of the remote center-of-motion RCM to the incision maker in accordance with stage S116 and S118 is omitted in view of the registration of the remote center-of-motion RCM to the incision point as illustrated in volume image during stage S110. Thus, upon the surgeon acknowledging the confirmation of stage S114, servo module 61a proceeds from stage S114 to stage S120 for an automatic servo control via servo module 61a of pitch actuator 41 and/or yaw actuator 43 as needed to axially align laser beam LB with the planned tool trajectory TT as shown in FIG. 1.

Based on the encoding tracking of stage S106 and registration of stage S110, for encoded active embodiments of robot platform 30 (“EARP”), a RCM alignment in accordance with stage S114 and a registration of the remote center-of-motion RCM to the incision maker in accordance with stage S116 and S118 are omitted in view of the registration of the remote center-of-motion RCM to the incision point as illustrated in volume image during stage S110. Thus, based on the platform tracking of stage S106 and the registration of stage S110, servo module 61a proceeds from stage S110 to stage S120 for an automatic servo control via servo module 61a of pitch actuator 41 and/or yaw actuator 43 as needed to axially align laser beam LB with the planned tool trajectory TT as shown in FIG. 1.

Still referring to FIGS. 1 and 2, upon a termination of flowcharts 80 and 100, those having ordinary skill in the art will appreciate the biopsy phase of FIG. 1 may be conducted in a precise, controller manner with minimal human error.

Referring to FIG. 1, in practice, the registration phase may be further automated and/or may utilize a variety of different optical end effectors.

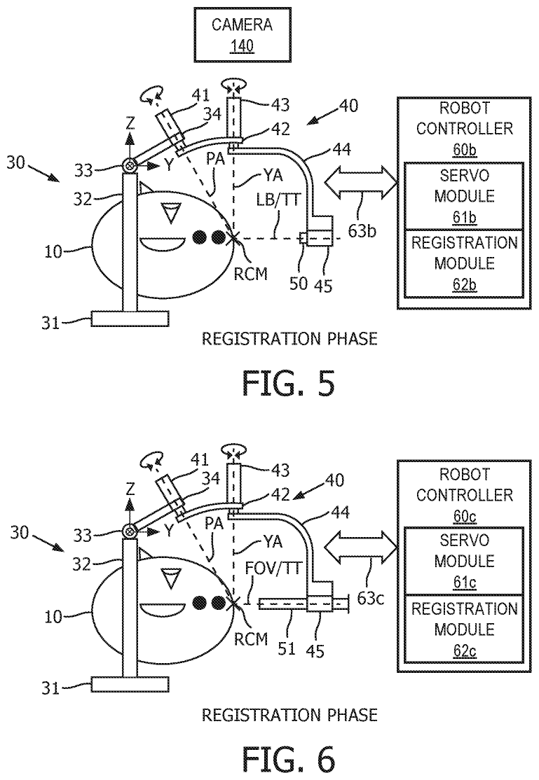

For example, FIG. 5 illustrates an exemplary registration phase of the present disclosure incorporating a camera 140 within the operating space (e.g., attached to robot platform 30 or concentric arc robot 40) in a manner that positions the markers attached to the head of patient 10 and laser pointer 50 within a field of view of camera 140. As such, a servo module 61b is structurally configured to communicate with a controller of camera 130 via communication path 63b to automatically perform the servo control of robot 30 when centering laser beam LB of laser pointer 50 on the markers as previously described herein. Such control eliminates any need for the surgeon to interface with servo module 61b during stage S108 and stage S118 (if applicable) as shown in FIG. 2.

Also by example, FIG. 6 illustrates an exemplary registration phase of the present disclosure utilizing an endoscope 51 alternative to laser pointer 50. For this embodiment, a servo module 61c is structurally configured to perform an automatic servo control that involves a centering of each marker within a field-of-view of endoscope 51 during stage S108 and stage S118 (if applicable) as shown in FIG. 2. More particularly, for markers 130-133 as shown respectively in FIGS. 3A-3D, servo module 61c performs the automatic servo control in a sequential centering markers 130-133 within the field-of-view of endoscope 51 stage S108 and stage S118 (if applicable) as shown in FIG. 2.

In practice, the controllers of FIG. 1 may be installed within a single workstation or distributed across multiple workstations.

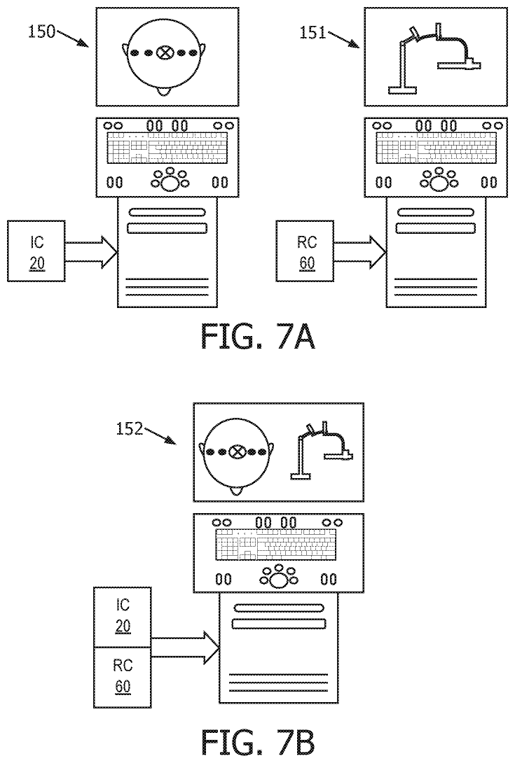

For example, FIG. 7A illustrates an imaging workstation 150 having an imaging controller of the present disclosure (e.g., imaging controller 20) installed therein for CT, MRI or US imaging, and a surgical robot workstation 151 having a robot controller of the present disclosure (e.g., robot controller 60) installed therein for executing a servo module and a registration module of the present disclosure.

By further example, FIG. 7B illustrates a workstation 152 having an imaging controller of the present disclosure (e.g., imaging controller 20) installed therein for CT, MRI or US imaging, and having a robot controller of the present disclosure (e.g., robot controller 60) installed therein for executing a servo module and a registration module of the present disclosure. For workstation 152, the controllers may physically/logically segregated or integrated.

Also in practice, a registration module of the present disclosure may be an application of an imaging controller of the present disclosure in communication with a robot controller of the present disclosure.

Referring to FIGS. 1-7, those having ordinary skill in the art will appreciate numerous benefits of the present disclosure including, but not limited to, a novel and unique optical registration of a remote center-of-motion robot to a patient for deployment of an interventional tool in a precise, controlled manner with minimal risk of human error.

Furthermore, as one having ordinary skill in the art will appreciate in view of the teachings provided herein, features, elements, components, etc. described in the present disclosure/specification and/or depicted in the FIGS. 1-9 may be implemented in various combinations of electronic components/circuitry, hardware, executable software and executable firmware and provide functions which may be combined in a single element or multiple elements. For example, the functions of the various features, elements, components, etc. shown/illustrated/depicted in the FIGS. 1-7 can be provided through the use of dedicated hardware as well as hardware capable of executing software in association with appropriate software. When provided by a processor, the functions can be provided by a single dedicated processor, by a single shared processor, or by a plurality of individual processors, some of which can be shared and/or multiplexed. Moreover, explicit use of the term “processor” should not be construed to refer exclusively to hardware capable of executing software, and can implicitly include, without limitation, digital signal processor (“DSP”) hardware, memory (e.g., read only memory (“ROM”) for storing software, random access memory (“RAM”), non-volatile storage, etc.) and virtually any means and/or machine (including hardware, software, firmware, circuitry, combinations thereof, etc.) which is capable of (and/or configurable) to perform and/or control a process.

Moreover, all statements herein reciting principles, aspects, and embodiments of the invention, as well as specific examples thereof, are intended to encompass both structural and functional equivalents thereof. Additionally, it is intended that such equivalents include both currently known equivalents as well as equivalents developed in the future (e.g., any elements developed that can perform the same or substantially similar function, regardless of structure). Thus, for example, it will be appreciated by one having ordinary skill in the art in view of the teachings provided herein that any block diagrams presented herein can represent conceptual views of illustrative system components and/or circuitry embodying the principles of the invention. Similarly, one having ordinary skill in the art should appreciate in view of the teachings provided herein that any flow charts, flow diagrams and the like can represent various processes which can be substantially represented in computer readable storage media and so executed by a computer, processor or other device with processing capabilities, whether or not such computer or processor is explicitly shown.

Furthermore, exemplary embodiments of the present disclosure can take the form of a computer program product or application module accessible from a computer-usable and/or computer-readable storage medium providing program code and/or instructions for use by or in connection with, e.g., a computer or any instruction execution system. In accordance with the present disclosure, a computer-usable or computer readable storage medium can be any apparatus that can, e.g., include, store, communicate, propagate or transport the program for use by or in connection with the instruction execution system, apparatus or device. Such exemplary medium can be, e.g., an electronic, magnetic, optical, electromagnetic, infrared or semiconductor system (or apparatus or device) or a propagation medium. Examples of a computer-readable medium include, e.g., a semiconductor or solid state memory, magnetic tape, a removable computer diskette, a random access memory (RAM), a read-only memory (ROM), flash (drive), a rigid magnetic disk and an optical disk. Current examples of optical disks include compact disk—read only memory (CD-ROM), compact disk—read/write (CD-R/W) and DVD. Further, it should be understood that any new computer-readable medium which may hereafter be developed should also be considered as computer-readable medium as may be used or referred to in accordance with exemplary embodiments of the present disclosure and disclosure.

Having described preferred and exemplary embodiments of novel and inventive optical registration of a remote center-of-motion robot to a patient (which embodiments are intended to be illustrative and not limiting), it is noted that modifications and variations can be made by persons having ordinary skill in the art in light of the teachings provided herein, including the FIGS. 1-7. It is therefore to be understood that changes can be made in/to the preferred and exemplary embodiments of the present disclosure which are within the scope of the embodiments disclosed herein.

Moreover, it is contemplated that corresponding and/or related systems incorporating and/or implementing the device or such as may be used/implemented in a device in accordance with the present disclosure are also contemplated and considered to be within the scope of the present disclosure. Further, corresponding and/or related method for manufacturing and/or using a device and/or system in accordance with the present disclosure are also contemplated and considered to be within the scope of the present disclosure.

1. A robotic surgical system for a minimally invasive procedure involving a planned tool trajectory through a planned incision point into a patient, the robotic surgical system comprising:
an optical end-effector;
a RCM robot operable to rotate the optical end-effector about a remote center-of-motion defined by a structural configuration of the RCM robot; and
a robot controller,
wherein the robot controller is operable in communication with the RCM robot to control an optical pointing by the RCM robot of the optical end-effector to at least one marker attached to the patient, and
wherein the robot controller is further operable in communication with the RCM robot to control an axial alignment by the RCM robot of the optical end-effector to the planned tool trajectory as illustrated within a volume image of the patient based on a registration of the remote center-of-motion to the planned incision point as illustrated within the volume image of the patient derived from an optical pointing by the RCM robot of the optical end-effector to the at least one marker attached to the patient.

2. The robotic surgical system of claim 1, wherein the RCM robot is a concentric arc robot having a pitch degree of freedom and a yaw degree of freedom for rotating the optical end-effector about a remote center-of-motion.

3. The robotic surgical system of claim 1, further comprising:
a robot platform operable to position the RCM robot relative to the patient;
wherein the robot controller is operable in communication with the RCM robot and the robot platform to control the optical pointing by the RCM robot and the robot platform of the optical end-effector to the at least one marker attached to the patient; and
wherein the robot controller is further operable in communication with the RCM robot and to the robot platform to control an axial alignment by the RCM robot and the robot platform of the optical end-effector to the planned tool trajectory as illustrated within the volume image of the patient based on the registration of the remote center-of-motion to the planned incision point as illustrated within the volume image of the patient derived from the optical pointing by the RCM robot of the optical end-effector to the at least one marker attached to the patient.

4. The robotic surgical system of claim 1,
wherein the optical end-effector is a laser pointer operable to emit a laser beam;
wherein the robot controller is operable in communication with the RCM robot to control an optically pointing by the RCM robot of an emission of the laser beam by the laser pointer to the at least one marker attached to the patient; and
wherein the robot controller is further operable in communication with the RCM robot to control an axial alignment by the RCM robot of the emission of the laser beam by the laser pointer to the planned tool trajectory as illustrated within the volume image of the patient based on the registration of the remote center-of-motion to the planned incision point as illustrated within the volume image of the patient derived from the optical pointing by the RCM robot the emission of the laser beam by the laser pointer to the at least one marker attached to the patient.

5. The robotic surgical system of claim 1,
wherein the optical end-effector is an endoscope having a field-of-view;
wherein the robot controller is operable in communication with the RCM robot to control an optical pointing by the RCM robot of the field-of-view of the endoscope to the at least one marker attached to the patient; and
wherein the robot controller is further operable in communication with the RCM robot to control an axially alignment by the RCM robot of the field-of-view of the endoscope to the planned tool trajectory as illustrated within the volume image of the patient based on the registration of the remote center-of-motion to the planned incision point as illustrated within the volume image of the patient derived from the optical pointing by the RCM robot of the field-of-view of the endoscope to the at least one marker attached to the patient.

6. The robotic surgical system of claim 1, further comprising:
a camera operable to image the optical end-effector relative to the at least one marker attached to the patient; and
wherein the robot controller is operable in communication with the RCM robot and the camera to control an optically point by the RCM robot of the optical end-effector to the at least one marker attached to the patient.

7. A robotic surgical method for a minimally invasive procedure involving a planned tool trajectory through a planned incision point into a patient, the robotic surgical method comprising:
a RCM robot optically pointing an optical end-effector to at least one marker attached to the patient;
a registration module deriving a registration of a remote center-of-motion to the planned incision point as illustrated within a volume image of the patient from the optical pointing by the RCM robot to the optical end-effector to the at least one marker attached to the patient,
wherein the remote center-of-motion is defined by a structural configuration of the RCM robot; and
the RCM robot axially aligning the optical end-effector to the planned tool trajectory as illustrated within the volume image of the patient based on the registration by the registration module of the remote center-of-motion to the planned incision point as illustrated within the volume image of the patient.

8. The robotic surgical method of claim 7,
wherein the at least one marker includes a plurality of markers;
wherein each marker of the plurality of markers has a unique shape.

9. The robotic surgical method of claim 7, further comprising:
a robot controller servo controlling the RCM robot for optically pointing the optical end-effector to the at least one marker attached to the patient and for axially aligning the optical end-effector to the planned tool trajectory as illustrated within the volume image of the patient.

10. The robotic surgical method of claim 9,
wherein the optical end-effector is operable to image the at least one marker attached to the patient; and
wherein the robot controller servo controls the RCM robot for optically pointing the optical end-effector to the at least one marker attached to the patient based on a correspondence of the at least one marker as illustrated in the volume image of the patient and as imaged by the optical end-effector.

11. The robotic surgical method of claim 7,
wherein the optical end-effector is a laser pointer emitting a laser beam;
wherein the RCM robot optically points the emission of the laser beam by the laser pointer to the at least one marker attached to the patient;
wherein the registration module derives the registration of the remote center-of-motion to the planned incision point as illustrated within the volume image of the patient from the optical pointing by the RCM robot of the emission of the laser beam by the laser pointer to the at least one marker attached to the patient; and
wherein the RCM robot axially aligns the emission of the laser beam by the laser pointer to the planned tool trajectory as illustrated within the volume image of the patient based on the registration by the registration module of the remote center-of-motion to the planned incision point as illustrated within the volume image of the patient.

12. The robotic surgical method of claim 7,
wherein the optical end-effector is an endoscope having a field-of-view;
wherein the RCM robot optically points the field-of-view of the endoscope to the at least one marker attached to the patient;
wherein the registration module derives the registration of the remote center-of-motion to the planned incision point as illustrated within the volume image of the patient from the optical pointing by the RCM robot of the field-of-view of the endoscope to the at least one marker attached to the patient; and
wherein the RCM robot axially aligns the field-of-view of the endoscope to the planned tool trajectory as illustrated within the volume image of the patient based on the registration by the registration module of the remote center-of-motion to the planned incision point as illustrated within the volume image of the patient.

13. The robotic surgical method of claim 7, further comprising:
a camera imaging the optical end-effector relative to the at least one marker attached to the patient; and
wherein RCM robot optically points the optical end-effector to the at least one marker attached to the patient based on the imaging by the camera of the optical end-effector relative to the at least one marker attached to the patient.

14. The robotic surgical method of claim 7, further comprising:
a passive robot platform positioning the RCM robot relative to the head of the patient.

15. The robotic surgical method of claim 14, further comprising:
a robot controller tracking the positioning by the passive robot platform of the RCM robot relative to the head of the patient.

16. The robotic surgical method of claim 7, further comprising:
an active robot platform positioning the RCM robot relative to the head of the patient.

17. The robotic surgical method of claim 16, further comprising:
a robot controller tracking the positioning by the active robot platform of the RCM robot relative to the head of the patient.

18. The robotic surgical method of claim 17, further comprising:
the RCM robot optically pointing the optical end-effector to a location of the head of the patient corresponding to a location of the planned incision point illustrated within the volume image of the patient.

19. The robotic surgical method of claim 18, further comprising:
attaching an incision marker to a location of the patient optically pointed to by the optical end-effector.

20. The robotic surgical method of claim 18, further comprising:
a RCM robot optically pointing an optical end-effector to at least one marker and the incision marker attached to the patient; and
wherein the registration module registers the remote center-of-motion to the planned incision point as illustrated within a volume image of the patient from the optical pointing by the RCM robot of the optical end-effector to the at least one marker and the incision marker attached to the patient.
